(19) United States
(12) Patent Application Publication
(10) Pub. No.: US 2022/0020483 A1
(43) Pub. Date: Jan. 20, 2022

(54) SMART ZONE PROTECTION SYSTEM FOR INFECTION CONTROL

(71) Applicant: Ahmad SALEH, Dubai (AE)

(72) Inventor: Ahmad SALEH, Dubai (AE)

(21) Appl. No.: 17/377,947

(22) Filed: Jul. 16, 2021

Related U.S. Application Data
(60) Provisional application No. 63/053,633, filed on Jul. 18, 2020.

(51) Int. Cl. G16H 40/63; G16H 40/20; G16H 50/30; G16H 50/80

(52) U.S. Cl. CPC G16H 40/63; G16H 50/80; G16H 50/30; G16H 40/20

(57) ABSTRACT

There is provided a smart zone protection system for infection control within a controlled zone, the smart zone protection system comprising (a) a plurality of smart face protective devices associated to various users; and (b) at least one zone advertising device for remote zone proximity detection and connection/pairing with the smart face protective devices. The smart face protective devices are configured to be wore by users and they comprise a face shield configured to be automatically (hands-free and without any human intervention) adjusted between an unsecured position and a secured position based on a zone notification signal received with an indication of the position of the user with respect to the controlled zone.

### CROSS-REFERENCE TO RELATED APPLICATIONS AND PRIORITY

The present invention claims priority from the previously filed U.S. Provisional Patent Application No. 63/053,633 filed Jul. 18, 2020. This patent application is herein incorporated by reference in its entirety.

### FIELD OF THE INVENTION

The present invention relates to personal protective equipment, and more particularly to a smart zone protection system for infection control (such as COVID-19) within a controlled zone (preferably an indoor zone), and a related method.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

The COVID-19 and other infectious diseases have changed the way people and businesses operate. Social distancing is nowadays a must in order to prevent the spread of such infectious diseases. Unfortunately, with the ease of lockdowns, businesses and governments are still struggling to find solutions for securing the health and safety of their people. Traditional face mask and face shields failed to provide an appropriate remedy to businesses, governments and facility managers for controlling infections in business and public places such as business offices, shopping malls, hospitals, schools, and so on. People are failing to appropriately apply the their masks or face shields on a voluntary basis or apply social distancing rules which is resulting on an increasing number of infections every day. There are certain zones which require a more attentive infection control for the infection risk associated to these places of example (such as highly crowded areas or narrow spaces with small distances between persons). Traditional personal equipment devices and systems fail to address these issues.

### SUMMARY OF THE INVENTION

As a first aspect of the invention, there is provided a smart face protective device to be wore by a user for infection control, where the face protective device comprises a face shield configured to be automatically (hands-free and without any human intervention) adjusted between an unsecured position and an unsecured position based on a zone notification signal received with an indication of the position of the user with respect to the controlled zone. In an embodiment of the invention, the face shield is adjusted from an unsecured position into a secured position based on a zone notification signal received with an indication of the user entering a controlled zone.

Preferably, the face shield is configured to be locked in place in the secured position as long as the user is within the physical boundaries of the controlled zone.

Preferably, the face shield is configured to be unlocked for enabling the user to manually remove/unsecure the face shield based on a determination that the user exited the controlled zone.

Preferably, the determination of the user exited the controlled zone is based on the absence of a signal received with an indication of the user is within the controlled zone.

Preferably, the determination of the user existed the controlled zone is based on a signal received with an indication of the user exiting the controlled zone.

Preferably, the smart face protective device has the structure and shape of a face shield device or a helmet device.

As a further aspect of the invention, there is provided a zone advertising device for zone proximity detection by a remote smart face protective device wore by a user for infection control, the zone advertising device comprising a transmitter for transmitting a zone notification signal to the smart face protective device indicative of a positioning of a user with respect to a controlled zone.

Preferably, the zone advertising device comprises a memory in connection with the transmitter for storing a unique identifier of the zone advertisement device, and wherein the zone notification signal comprises said unique identifier of the zone advertisement device which is used by the smart face protective device for the authentication of the zone notification signal.

Preferably, the positioning of a user with respect to a controlled zone is a presence/proximity of a controlled zone, presence/proximity of an entry point of a controlled zone, or presence/proximity of an exit point to a controlled zone.

Preferably, the transmitter comprises at least one of a radio transmitter, an ultrasound transmitter and a BLE transmitter.

Preferably, the zone advertising device a beacon device periodically and continuously sending zone notification signals during operation and the smart face protective device continuously and periodically scanning for capturing and processing the zone notification signals.

As a further further aspect of the invention, there is provided a smart zone protection system for infection control within a controlled zone, the smart zone protection system comprising:
a plurality of smart face protective devices associated to various users for infection control in accordance with the various embodiments of the present invention; and
at least one zone advertising device for remote zone proximity detection and connection/pairing with the smart face protective devices in accordance with the various embodiments of the present invention.

Preferably, the controlled zone is an indoor facility such as a hospital, a school, a conference room, a reception area, an elevator, a building waiting area, a shopping mall, and a corporation hallway.

As another aspect of the invention, there is provided a method of controlling infections within a controlled zone, the method comprising:
configuring at least one zone advertising device according to the various embodiments of the invention in such a manner to have a strong signal coverage within the entire controlled zone; and
configuring or allowing to be configured a plurality of smart face protective devices associated to various users in accordance with the various embodiments of the invention for enabling remote zone proximity detection and connection/pairing with the at least one zone advertising device.

Preferably, the configuring or allowing to be configured a plurality of smart face protective devices comprise configuring the smart face protective devices with the unique identifiers associated to the at least one zone advertising device and the signal strength threshold.

Preferably, the method of controlling infections within a controlled zone comprises deploying the at least one zone advertising device within the controlled zone configured to emit zone notification signals and configuring the smart face protective devices to automatically secure and lock in place the face shields when said zone notifications signals are detected in accordance with the preconfigured criteria.

Preferably, the method of controlling infections within a controlled zone, comprising:
deploying a first zone advertising device at the entry point of the controlled zone and a second zone advertising device at the exit point of the controlled zone;
configuring the first zone advertising device to emit zone entry point notification signals and configuring the second zone advertising device to emit zone exit point notification signals;
configuring the smart face protective devices to automatically secure and lock in place the face shields when said zone entry point notifications signals are detected in accordance with preconfigured criteria; and
configuring the smart face protective devices to automatically unsecure or unlock the face shields when said zone exit point notifications signals are detected in accordance with preconfigured criteria.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which:

FIG. 1 is a block diagram illustrating a Smart Zone Protection System (SZPS) for infection control within a controlled zone, the system comprising one zone proximity detector in communication with smart face protective devices (SFPDs) in accordance with an embodiment of the present invention;

FIG. 2 is a block diagram illustrating a Smart Zone Protection System (SZPS) for infection control within a plurality of controlled zones, the system comprising a plurality of respective zone proximity detectors in communication with SFPDs in accordance with an embodiment of the present invention;

FIG. 3 is a block diagram illustrating an advertising device in communication with a smart face protective device (SFPD) in accordance with an embodiment of the present invention;

FIG. 4 illustrates a SFPD Face Shield Unit in accordance with an embodiment of the present invention;

FIG. 5 illustrates the use of beacon devices as advertising devices in accordance with an embodiment of the present invention;

FIG. 6 illustrates a controlled zone deploying a plurality of zone advertising devices designed to be in communication with smart face protective devices for activating/securing their associated face shields according to an embodiment of the present invention;

FIG. 7 illustrates a controlled zone deploying an entry point advertising device and an exit point advertising device designed to be in communication with smart face protective devices for respectively activating/securing and deactivating/unsecuring their associated face shields according to an embodiment of the present invention;

FIG. 8 illustrates a group of persons wearing smart face protective devices with activated/secured face shields within a controlled zone deploying a smart zone protection system having active beacon devices in accordance with an embodiment of the present invention;

FIG. 9 illustrates a process of controlling infections within a zone (controlled zone) in accordance with an embodiment of the present invention;

FIG. 10 is a block diagram illustrating a Smart Access Control System (SACS) for infection control in accordance with an embodiment of the present invention;

FIG. 11 illustrates a group of persons wearing smart face protective devices designed to be in communication with a smart access control system (SACS) for controlling access to a gated/controlled zone in accordance with an embodiment of the present invention;

FIG. 12 illustrates a process of controlling access to a gated/controlled zone for infection control in accordance with an embodiment of the present invention;

### DETAILED DESCRIPTION OF THE INVENTION

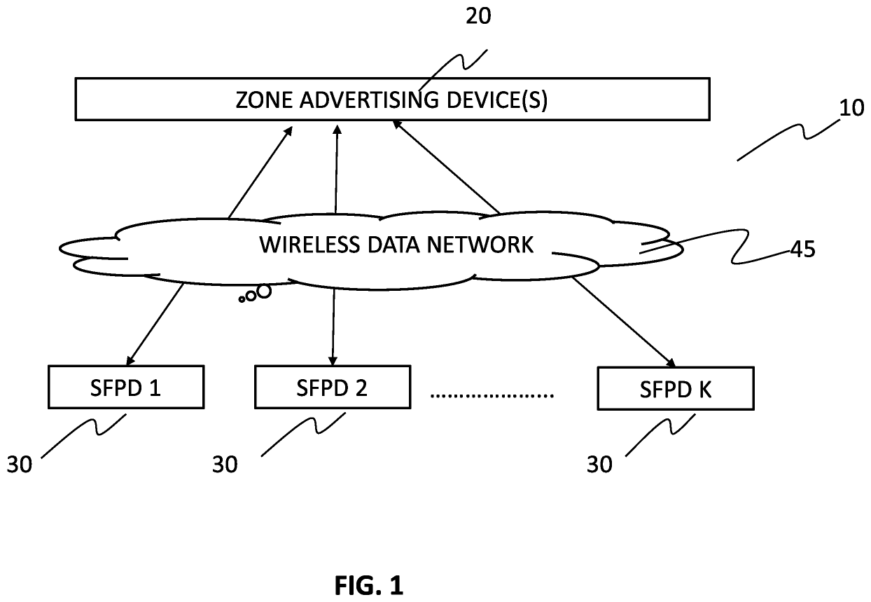

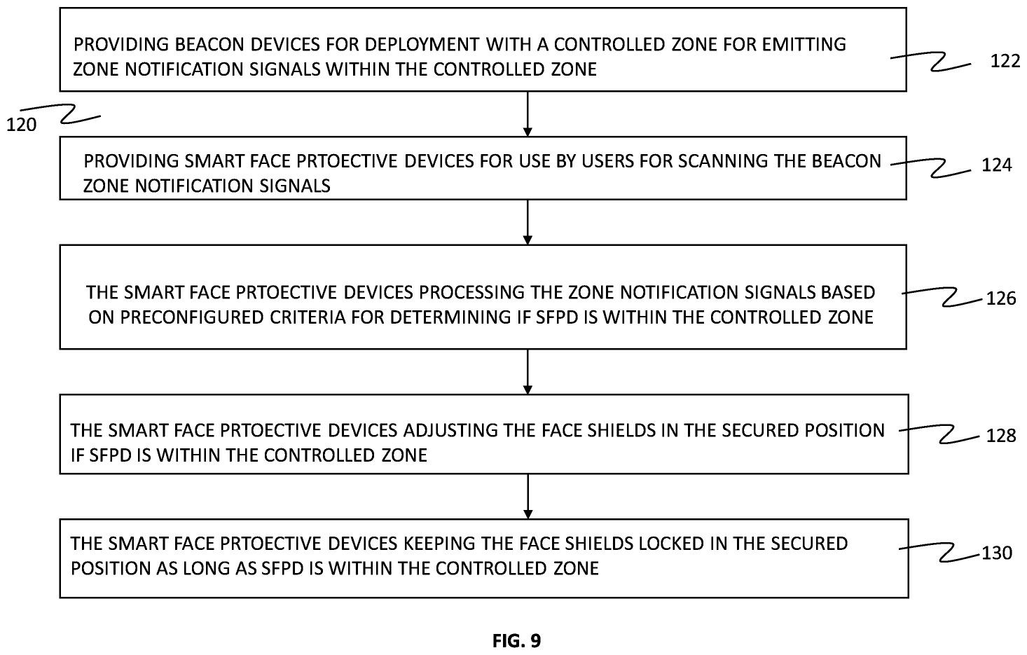

The aspects of the invention will be described in conjunction with FIGS. 1-9. In the Detailed Description, reference is made to the accompanying figures, which form a part hereof, and in which is shown by way of illustration specific embodiments in which the invention may be practiced. It is to be understood that other embodiments may be utilized and logical changes may be made without departing from the scope of the present invention. The following detailed description, therefore, is not to be taken in a limiting sense, and the scope of the present invention is defined by the appended claims.

Referring to FIG. 1-7, there is provided a Smart Zone Protection System (SZPS) 10 for controlling spread of infections in a controlled zone 15. The smart zone protection system 10 comprises one or more zone advertising devices 20 configured to be in communication with smart face protective devices 30 associated to users for automatically controlling the smart face protective devices 30 to be in secured positions when these are inside/within the controlled zone 15.

In an embodiment of the invention, the smart face protective device 30 is configured to be wore by a user for preventing or reducing spread of infectious droplets through a portal portion of the user's face, where the smart face protective device 30 is configured to be automatically adjusted (hands-free and without user intervention while being wore by the user) in the secured position based on a signal received from the one or more advertising device 20 which is indicative of the presence of the user (wearing the smart face protective device 30) within the controlled zone 15. The secured position is a position in which the portal portion of the user's face (eyes, nose and mouth) is isolated from the external environment for blocking spread of splashes and sprays between the user and other individuals (or objects within the controlled zone) through said portal portion and for blocking hands or other members or objects from reaching to said portal portion of the face.

In an embodiment of the invention, the zone advertising device 20 is configured to transmit zone notification signals notifying the smart face protective devices 30 about the presence/proximity of the controlled zone 15. The smart face protective devices 20 are configured to scan and process the zone notification signals, and based on said processing, the smart face protective devices 20 determine whether they are within the physical boundaries/proximity of the controlled zone 15. The smart face protective devices 20 are configured to be automatically (hands-free and without any user intervention) adjusted in the secured position (in such a way to cover the user's portal portion of the face to prevent the spread of infectious droplets or splashes between the user and others persons or objects) when the user is determined to be within the physical boundaries/proximity of the controlled zone 15.

In an embodiment of the invention, the zone advertising device 20 is configured to send zone notification signals on a periodic or regular basis and the smart face protective devices 20 are configured to continuously scan for such signals, capture and process these zone notification signals for determining the proximity of the smart face protective devices 30 with respect to the controlled zone 15. If a smart face protective device 30 is determined to be within the physical range/proximity of the controlled zone 15 (which can be determined based on the strength of the signal received for example), then the smart face protective device 30 is configured to be automatically adjusted in the secured position to cover the user's face for restricting the spread of any infections.

In an embodiment of the invention, the zone advertising device 20 keeps transmitting signals periodically to notify its presence to the smart face protective devices 30 and the smart face protective devices continuously scan for capturing any such zone notification signals and connect therewith when the zone notification signal received is compliant to the requirement as predefined by the smart face protective devices.

In an embodiment of the invention, the zone advertising devices 20 act as beacons, and the smart face protective devices 30 act as scanning devices. The zone advertising devices 20 periodically send zone notification signals, and smart face protective devices 30 continuously scan these signals. When a smart face protective device 30 approaches an advertising device 20, the zone notification signal strength increases in value. The smart face protective device 30 is configured to compare the strength of the zone notification signal received to the signal strength threshold as predefined by the smart face protective device 30. If the strength of the zone notification signal received is above the predefined signal strength threshold, then the smart face protective device 30 comes to a determination that the user is inside the controlled zone 15 and automatically puts the smart face protective device 30 in the secured position. This is done hands-dree and without any user intervention.

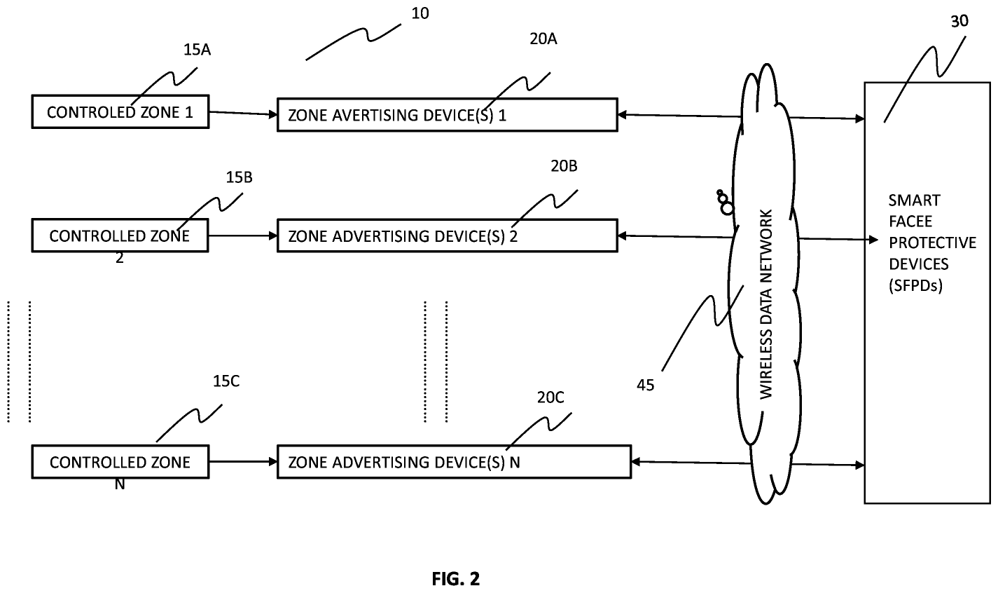

Referring to FIG. 2, in an embodiment of the invention, the smart zone protection system 10 is configured to be deployed in a facility having a plurality of zones to be controlled from infections. In this scenario, each controlled zone (15A, 15B, . . . , 15N) is being equipped respectively with a set of one or more zone advertising devices (20A, 20B, . . . , 20N) configured to emit zone notification signals within their respective zones. The exact number and positioning of the zone advertising devices 30 within the controlled zones 15 are determined on a case by case basis depending on the characteristics and typography of each zone in terms of dimensions, shape, environment, obstacles and so on. For example, a zone having a small area (3-4 meters) might be covered by a single zone advertising device 30, whereas larger zones might require more than one zone advertising device 30 capable of providing the required signal coverage to the entire zone. It is important that the zone notification signals maintain a certain strength threshold within the entire physical boundaries of the desired controlled zone 15 for enabling the smart face protective devices 20 within the controlled zone 15 to operate effectively. This is as week zone notification signals below the predefined signal strength threshold will be ignored by the smart face protective device 30.

The zone notification signals are configured to have suitable signal characteristics, including frequency and power, capable of being detected by the SFPDs while these are within the physical boundaries of the controlled zone 15.

In an embodiment of an invention, there is provided one or more zone advertising devices 20 deployed to provide signal coverage within the controlled zone 15 and the smart face protective device 30 is configured to continuously scan and process the received zone notification signals, and to maintain the secured position in place as long as the two devise 20 and 30 are paired/connected to each other.

In another embodiment of the invention, there is provided a first group of one or more zone advertising devices 20M at the entry point (s) of the controlled zone 15, and a second group of one or more zone advertising devices 20N at the exit point (s) of the controlled zone. According to this embodiment, the first group of zone advertising devices 20M are configured to emit zone entry point notification signals as indication to the entrance of the user within the controlled zone 15 and the second group of zone advertising devices 20N are configured to emit zone exit point notification signals as an indication of the exit of the user from the controlled zone 15. The smart face protective devices 30 are configured to be automatically adjusted into the secured position (and locked in position) when they enter the controlled zone 15 (when they detect and process the zone entry point notification signals while passing through the entry point (s) of the controlled zone 15), and to be automatically unlocked from the secured position (or adjusted into the unsecured position) when pass through the exit point (s) of the controlled zone 15 (when they detect and process the zone exit point notification signals while passing through the exit point (s) of the controlled zone 15). The automatic adjustment is conducted hands-free without any human intervention. To determine if the smart face protective device 30 is passing through an entry point or an exit point of a controlled zone, the smart face protective device 30 scans for the signals and determine whether these signals are zone entry point notification signals or, alternatively, whether they are zone exit point notification signals. This determination can be based on the information received in the signal itself or based on any characteristic thereof such as the frequency or power in view of predefined criteria known a priori and stored at the smart face protective device 30.

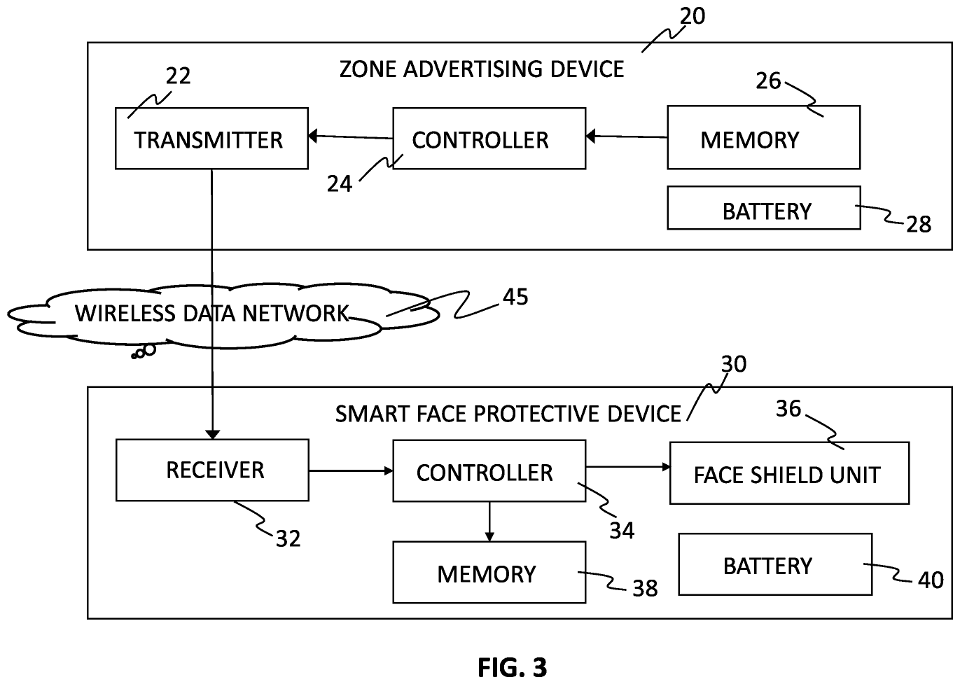

Referring to FIG. 3, in an embodiment of the invention, the zone advertising device 20 comprises a transmitter 22, a controller 24, a memory 26 and a battery 28 for providing power supply to the device 20. The memory 26 stores a unique identifier enabling to uniquely identify the zone advertising device 20 and/or to the controlled zone 15 to which the zone advertising device 20 is being associated. The controller 24 is connected to the memory 26 and to the transmitter 22 for retrieving the zone advertising device unique identifier from the memory 26 and instructing the transmitter 22 for emitting zone notification signals comprising the zone advertising device unique identifier.

In an embodiment of the invention, the smart face protective device 30 comprises a receiver 32, a controller 34, a face shield unit 36, a memory 38 and a battery 40 for providing power supply to the device 30. In an embodiment of the invention, the face shield unit 36 comprises a face shield operation module 160, a lock/unlock mechanism 162, a motor 164, and face shield 168 configured to be automatically (hands-free and without human intervention) closed/activated for securely shielding the portal portion of the user's face when the face shield 168 is in the secured position, and to be open/deactivated when the face shield 168 is in the unsecured positon. According to this embodiment, the face shield 168 is used to act as a barrier between the user's portal portion of the face and the external environment.

In an embodiment of the invention, adjusting the face shield 168 into a secured position comprises closing the face shield 168 such that the user's face (or at least the portal portion) is protected and isolated from the external environment to avoid any fluid communication of droplets or splashes with other persons and to restrict the user's hands of reaching to the face (or at least the portal portion of the face). In an embodiment of the invention, adjusting the face shield 168 into an unsecured position comprises opening the face shield 168 such that the face of the user is exposed to the external environment.

The face shield 168 is made of a suitable material depending on the application. In an embodiment of the invention, the face shield 168 is made of a solid material such as transparent plastic or glass acting as barrier to droplets and body fluids without obstructing the vision of the user.

In an embodiment of the invention, the face protective device 30 has a unique identifier allowing to uniquely identify the face protective device 30 and/or associated user wearing the device 30. In an embodiment of the invention, the face protective device 30 is associated to a specific user and the unique identifier enables to uniquely identify the user associated to the face protective device. In an embodiment of the invention, the unique identifier is stored inside the memory 38 of the face protective device 30 and accessed to/used by the controller 34 depending on the application. The unique identifier can be static or dynamic such that it can be updated to reflect the identity of the user using the face protective device 30 at any given time. In an embodiment of the invention, the input/output interface of the face protective device is configured to enable the set up/change of the unique identifier based on the identity of the user using the device 30.

In an embodiment of the invention, the face shield unit 36 comprises a motor 164 (preferably an electric motor) connected to the face shield 168 for opening and closing the face shield 168 (or at least a portion of the face shield responsible of protecting the portal portion of the user's face) as a function of the zone notification signals received from the zone advertising device 30 after being processed by the controller 34 as a function of preconfigured criteria such as the signal strength threshold (and possibly the received zone advertising device unique identifier).

In an embodiment of the invention, the memory 38 of the smart face protective device 30 stores the unique identifiers associated to the all the approved zone advertising devices 20. This is as the smart face protective device 30 would not process any signals received from unapproved zone advertising devices 20. This is in order to distinguish the zone notification signals received from the zone advertising devices 20 from other signals originating from other types of devices. According to this embodiment, the controller 34 compares the received unique identifier of the zone advertising device 20 to the pre-stored unique identifier inside the memory 38 to determine if there is match. If there is a match and the signal strength is above the predefined signal strength threshold, then the controller 34 sends an activation signal to the face shield unit 36 for adjusting the face shield in the secured position.

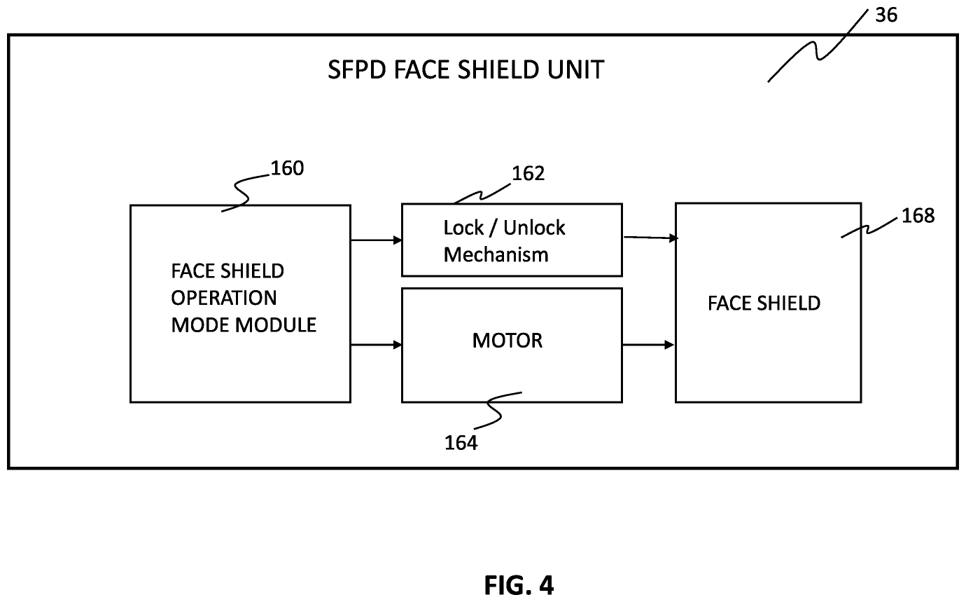

Referring to FIG. 4, the face shield unit 36 further comprises a lock/unlock mechanism 162 and a motor 164 connected to a face shield operation mode module 160 and to the face shield 168. The lock/unlock mechanism 162 comprises a mechanical or an electromagnetic clutch for locking the face shield 168 in a given position (insecure or secure position) which is also adapted to be deactivated to return the face shield in an unlocked position. The lock/unlock mechanism 162 obtains the lock/unlock instructions from the face shield operation mode module 160.

The motor 164 is preferably an electrical motor connected to the face shield 168 for automatically adjusting the face shield between a secure position (for example by closing the face shield) and an insecure position (for example by opening the face shield). When the face shield operation mode is set to be automatic, the motor 164 automatically adjusts the face shield 168 between a secured and unsecured position depending on whether the smart face protective device 30 is inside a controlled zone 15 as instructed by the controller 34. When the face shield operation mode is set to be manual, the motor 164 is receptive to a manual instruction by the user to either put the face shield 168 in a secured position (by closing the face shield for example) or to put it in the unsecured position (by opening the face shield 168 for example). In an embodiment of the invention, the face shields 168 are automatically locked in the secured position when the user is inside the controlled zone 15 in the sense that the user will not be able to insecure/open the face shields manually. Face shields are unlocked and brought back to a manual operation only when the user is outside the controlled zone 15.

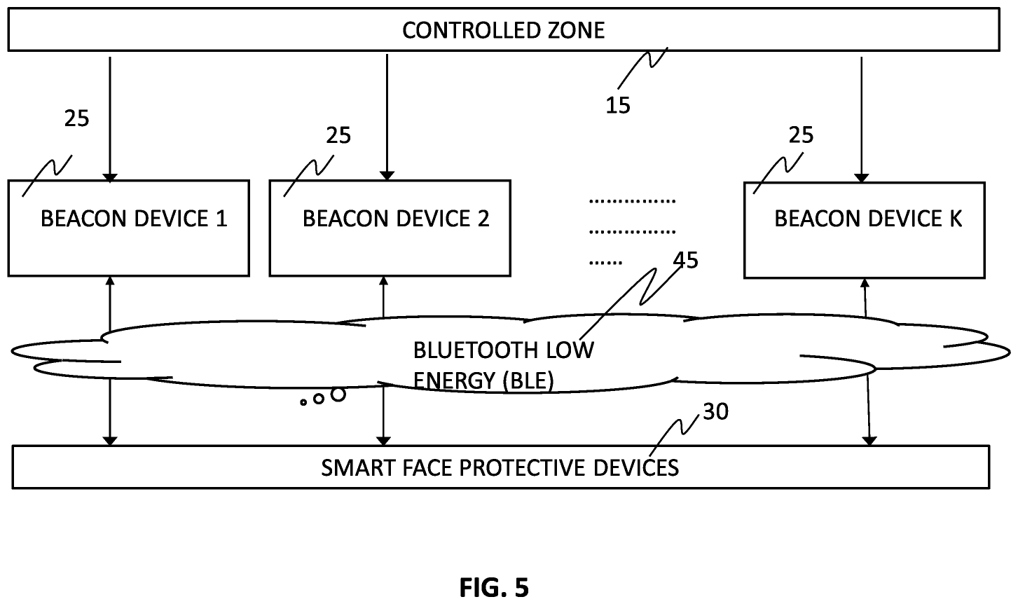

Referring to FIG. 5, in an embodiment of the invention, the zone advertising devices 20 are beacon devices 25 operating with a Bluetooth Low Energy (BLE) technology, and the receivers 32 of the smart face protective devices 30 are BLE operating receivers configured to be connected to the transmitters of the beacon devices 25. The beacon devices 25 are configured to be connected to the smart face protective devices 30 through a BLE data network 45. In an embodiment of the invention, the proximity zone detection technique is based on the strength of the received signal, the time window of the received signal, frequency of the received signal. BLE offers for a low cost, low energy and flexibility which may be a suitable technology to be used.

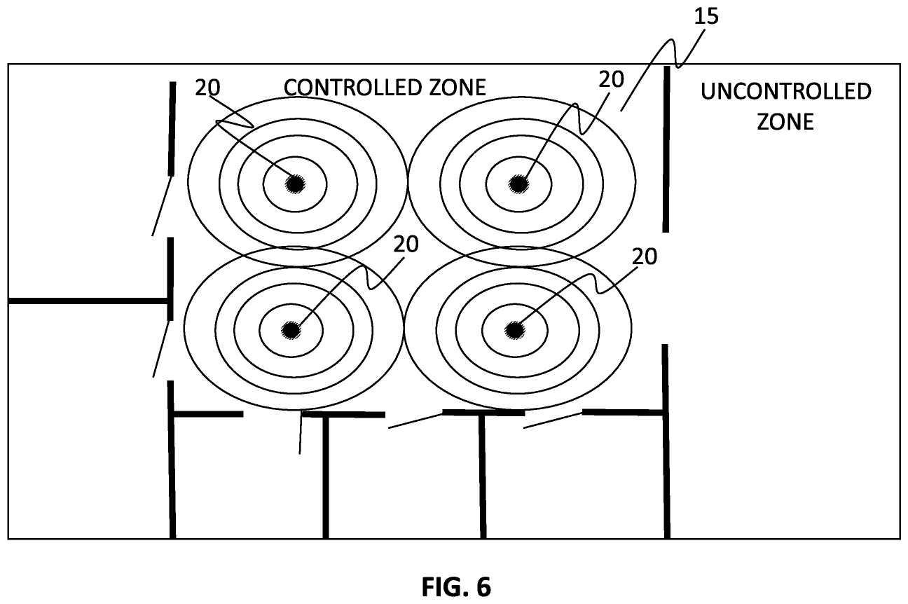

FIGS. 6-8 illustrate various examples of application of a smart zone protection system 10 in accordance with various embodiments of the present invention.

In an embodiment of the present invention, as illustrated in FIG. 6, zone advertising devices 20 are deployed within the controlled zone 25 and they are configured to emit zone notification signals having a signal strength above the predefined signal strength within the entire area of the controlled zone 25. The smart face protective devices 30 are configured in this case to continuously scan for zone notification signals and automatically (hands-free and without any human intervention) secure (and preferably lock) the face shields 168 in place as long as the user is within the controlled zone 25 according to a continuous scanning, processing of the received zone notification signals. The smart face protective devices 30 are configured to unlock the face shields 168 (from the secured position) or automatically adjust it in the unsecured position only when the user exits the controlled zone 25 which is determined absence receipt of zone notification signals according to the predefined criteria stored inside the smart face protective devices 30.

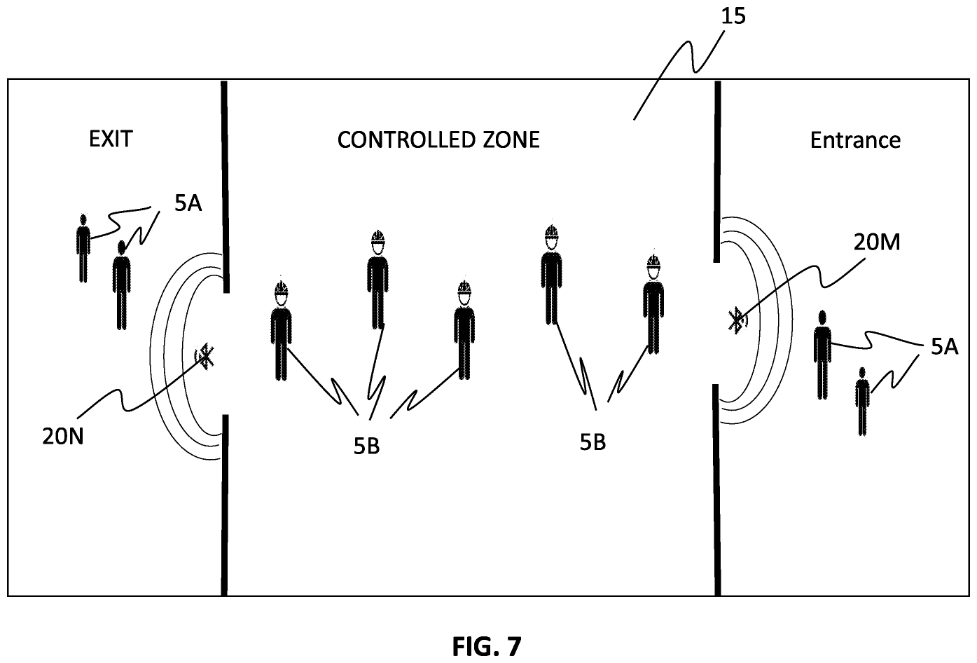

In an embodiment of the invention, as illustrated in FIG. 7, the controlled zone 25 can be secured through the entry and exit points of the controlled zone 25. In this case, it might not be required to deploy zone advertisement devices 20 within the controlled zone 25 itself. According to this arrangement, one or more zone entry point advertising devices 20M can be deployed at the entry point of the controlled zone 25, and one or more zone exit point advertising devices 20N can be deployed at the exit point of the controlled zone 25. The zone entry point advertising devices 20M transmit zone entry point notification signals indicating entrance to a controlled zone 25. The zone exit point advertising devices 20N transmit zone exit point notification signals indicating exit from a controlled zone 25. The smart face protective devices 30 are configured to receive and process these signals for adjusting the face shields 168 in the secured position or the unsecured position depending on the nature of the signal (entry point notification signal or exit point notification signal).

The smart face protective device 30 automatically adjusts the face shield in the secured position when a zone entry point notification signal is received according to the predefined criteria. This is in order to have the user secured with the face shield 168 in the secured position (and preferably locked in place) before entering the controlled zone 25. Preferably, the smart face protective device 30 is configured to keep the face shield 168 locked in place in the secured position as along as it does not receive an exit point notification signal from the zone exit point advertising device 20N. The smart face protective device 30 automatically unlocks the face shield 168 (from the secured position) or adjusts the face shield to the unsecured position when a zone exit point notification signal is received according to the predefined criteria.

Preferably, the entry and exit point notification signals received comprise the unique identifiers of the entry point and exit point advertising devices respectively 20M and 20N which are taken into consideration while processing these signals by the smart face protective device 30. The smart face protective device 30 determines the nature of the signal (whether it is an entry or exit point notification signal and in consequence the nature of the action to be conducted—securing/locking the face shield 168 or deactivating/unlocking the face shield 168) based on the unique identifiers received after comparison with the pre-stored unique identifiers. In an embodiment of the invention, the smart face protective device 30 stores (through the memory 38) the unique identifiers of the entry point and exit point advertising devices 20M and 20N with an indication of the nature of the zone advertising devices they represent (entry or exit) 20M or 20N and/or nature of the action required to be taken (adjust the face shield in the secured position or otherwise). The users within the controlled zone 25 (users 5B) have their face shields 168 secured in place (and preferably locked) as long as they are within the controlled zone 25. When the users exist the controlled zone (users 5A), the face shield is deactivated/unlocked.

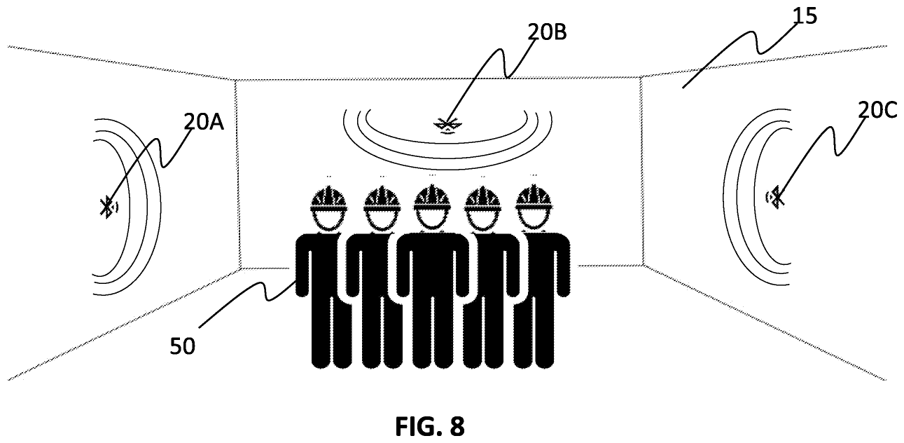

In an embodiment of an invention, referring to FIG. 8, the controlled zone 25 is a closed space having a single entry/exit point, such as an elevator. A possible arrangement to have one or more zone advertising devices 20 are positioned such that the signal coverage is omnipresent within the entire controlled zone 25 (preferably mounted within the controlled zone 25). The smart face protective devices 30 are configured to automatically (hands-free and without human intervention) adjust the face shields 168 into the secured positions (and preferably lock them in place such that the users cannot manually unsecure the face shields) when the users are located within the controlled zone 25 (elevator for example) and will be deactivated/unlocked when the users leave the controlled zone 25. Another possible arrangement is to have one or more zone entry/entry point advertising device at the entry point of the controlled zone, however the smart face protection devices 30 shall be configured to distinguish between the users entering and the users existing the controlled zone 25.

In an embodiment of the present invention, the zone advertising devices 20 comprise receivers in communication with the device controller 24 for receiving and keeping in record (storing) inside the device memory 26 the unique identifiers associated to the users/smart face protective devices 30 which were present within/in proximity of the controlled zone 25 within a given period of time. This can be determined based on the identity of the smart face protective devices 30 which have paired/connected to the zone advertising device 20 during that given time period. This will allow an administrator to track the users who have passed through the controlled zone 25 for any administrative or management purpose (such as health management or control).

In an embodiment of the invention, the transmitters 22 of the zone advertising devices 20 comprise at least one of radio transmitters emitting radio signals, ultrasound transmitters emitting ultrasound signals, and BLE transmitters emitting Bluetooth compatible signals. In this case, the receivers 32 of the smart face protective devices 30 comprise at least one radio receivers detecting radio signals, ultrasound receivers detecting ultrasound signals, and BLE receivers detecting Bluetooth compatible signals. In an embodiment of the invention, the transmitters 22 of the zone advertising devices 20 comprise radio transmitters emitting radio signal, and in the case the receivers 32 of the smart face protective devices 30 comprise radio receivers. In another embodiment, the transmitters 22 of the zone advertising devices 20 comprise ultrasound transmitters, and in the case the receivers 32 of the smart face protective devices 30 comprise ultrasound receivers. In yet another embodiment of the invention, the transmitters 22 of the zone advertising devices 20 comprise BLE transmitters emitting Bluetooth compatible signals, and in the case the receivers 32 of the smart face protective devices 30 comprise Bluetooth compatible receivers.

As another aspect of the invention, there is provided a process of controlling infections within a zone (controlled zone) in accordance with an embodiment of the present invention. The process comprises providing beacon devices for deployment within a controlled zone for emitting zone notification signals within the physical boundaries of the controlled zone (122), providing smart face protective devices for use by users where the smart face protective devices are configured to detect/scan the zone notification signals (124), where the smart face protective devices processing the received zone notification signals based on preconfigured criteria for determining if the SFPD is within the controlled zone (126), the smart face protective devices adjusting the face shields in the secured position if SFPD is within the controlled zone (128), and the smart face protective devices keeping the face shields locked in the secured position as long as SFPD is within the controlled zone (130).

The SZPS 10 can be used to control any geographical or physical zone of any type, including any indoor or outdoor area, depending on the application and needs. The controlled zone 15 can be any outdoor or indoor geographical or physical zone, space, area, room or infrastructure. Preferably, the smart zone protection system 10 is being deployed in an indoor environment.

In an embodiment of the invention, the controlled zone 15 is an indoor physical space requiring control for preventing the spread of infections between individuals while they are within that space.

The controlled zone 15 can for example be a geographical/physical location or space representing a high risk of contamination between people, such as an area with frequent crowds and/or narrow spacing between individuals such as an elevator, a store, a conference room, a pass hall in a private company or in a public area such as shopping mall, a school, a playground for kids, a reception area and so on. In an embodiment of the invention, the controlled zone is an area representing a high risk environment for the spread of infectious disease such as a hospital or a medical clinic for example.

In an embodiment of the invention, the controlled zone is a facility hosting people with vulnerable health conditions such as a healthcare facility hosting patients with vulnerable health conditions such elder patients or patients having chronic diseases such as cancer. Snice contamination of these patients can lead to very serious health consequences, securing the facilities where these vulnerable patients stay becomes very important.

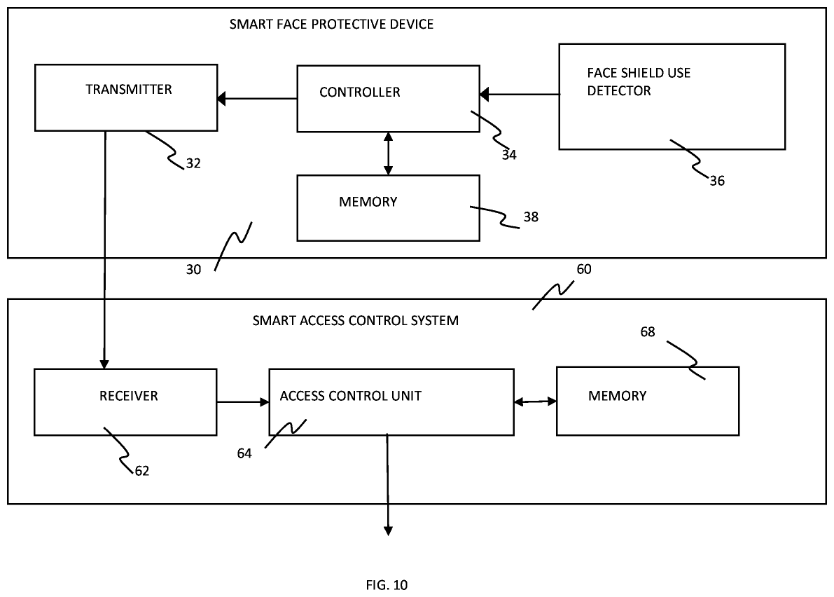

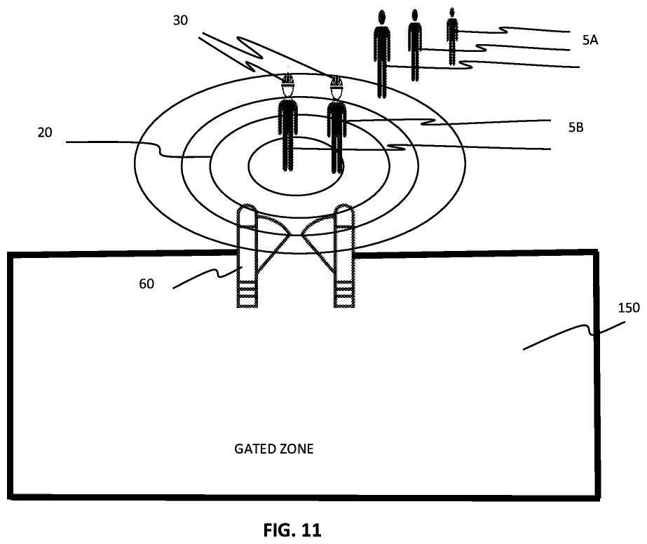

Referring to FIG. 10 and FIG. 11, there is provided a Smart Access Control System (SACS) for infection control in accordance with an embodiment of the present invention. The SACS 60 is configured to grant access to a gated zone 150 to users wearing the smart face protective device 30 only and to reject access to others. The SACS 60 comprises a receiver 62 configured to receive from the transmitter 32 of the SFPD 30 the unique identifier associated to the SFPD 30 (and preferably along with an indication on whether the SFPD 30 is being worn by the user). The Access Control Unit 64 of the SACS 60 is configured to determine based on the information received from the SFPD 30 whether the user is a registered user within the database of the SACS 68. If it is the case, access is granted and an instruction is sent by the Access Control Unit 64 to the gate or other access device.

The SFPD 30 comprises a face shield use detector 36 configured to determine if the face shield is being secured by the user and to send a shield use signal to the controller 34. The shield use signal is a signal either indicating that the SFPD 30 is being worn by a user and/or a signal indicating that the face shield is adjusted in the secured position. In an embodiment of the invention, the SFPD controller 34 sends to the SACS through the SFPD transmitter 32 an access request for granting access to the user of the SFPD 30. The access request comprises the SFPD/user unique identifier. Preferably, the access request also comprises an indication on whether the SFPD 30 is being worn by the user. This information is obtained from the face shield use detector 36 which is configured to detect and determine whether the SFPD 30 is being worn by the user and/or whether the face shield is being secured in the secure position. The SACS 60 is configured to reject access if the access request received does not comprise an indication that the SFPD 30 is being worn and/or face shield secured by the user. In an embodiment of the invention, the SFPD 30 does not send an access request to the SACS unless the SFPD is being worn and/or face shield is being secured by the user.

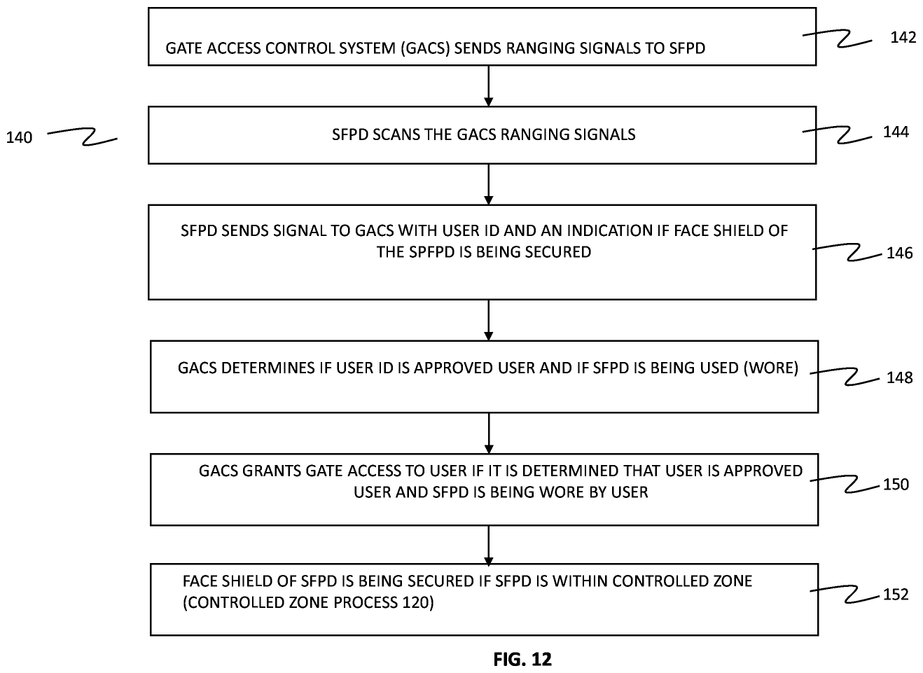

Referring to FIG. 12, there is provided a process of controlling access to a gated/controlled zone for infection control in accordance with an embodiment of the present invention comprising the steps of: the GACS sends gated zone notification signals on a continuous and period manner (142), the SFPD scans for and processes the gated zone notification signals (144), the SFPD sends an access request signal to the GACS with the SFPD ID (or user ID) and a confirmation that the SFPD is worn by the user (and/or face shield is being secured depending on the application) (146), the GACS grants access to the user if the user is determined to be an approved/registered user based on the user ID provided and if the SFPD is being worn by the user (148). In an embodiment of the invention, the face shield of the SFPD is being secured automatically when the user accesses a secured zone in accordance with the controlled zone access process (120).

Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims which follow.

1. A smart face protective device to be wore by a user for infection control, where the face protective device comprises a face shield configured to be automatically (hands-free and without any human intervention) adjusted between an unsecured position and an unsecured position based on a zone notification signal received with an indication of the position of the user with respect to the controlled zone.

2. The smart face protective device as claimed in claim 1, wherein the face shield is configured to be locked in place in the secured position as long as the user is within the physical boundaries of the controlled zone.

3. The smart face protective device as claimed in claim 2, wherein the face shield is configured to be unlocked for enabling the user to manually remove/unsecure the face shield based on a determination that the user exited the controlled zone.

4. The smart face protective device as claimed in claim 3, wherein the determination of the user exited the controlled zone is based on the absence of a signal received with an indication of the user is within the controlled zone.

5. The smart face protective device as claimed in claim 3, wherein the determination of the user existed the controlled zone is based on a signal received with an indication of the user exiting the controlled zone.

6. The smart face protective device as claimed in claim 1 having the structure and shape of a face shield device or a helmet device.

7. A zone advertising device for zone proximity detection by a remote smart face protective device wore by a user for infection control, the zone advertising device comprising a transmitter for transmitting a zone notification signal to the smart face protective device indicative of a positioning of a user with respect to a controlled zone.

8. The zone advertising device as claimed in claim 7, comprising a memory in connection with the transmitter for storing a unique identifier of the zone advertisement device, and wherein the zone notification signal comprises said unique identifier of the zone advertisement device which is used by the smart face protective device for the authentication of the zone notification signal.

9. The zone advertising device as claimed in claim 7, wherein the positioning of a user with respect to a controlled zone is a presence/proximity of a controlled zone, presence/proximity of an entry point of a controlled zone, or presence/proximity of an exit point to a controlled zone.

10. The zone advertising device as claimed in claim 7, wherein the transmitter comprises at least one of a radio transmitter, an ultrasound transmitter and a BLE transmitter.

11. The zone advertising device as claimed in claim 7, wherein the zone advertising device is a beacon device periodically and continuously sending zone notification signals during operation and the smart face protective device continuously and periodically scanning for capturing and processing the zone notification signals.

12. A smart zone protection system for infection control within a controlled zone, the smart zone protection system comprising:
a plurality of smart face protective devices associated to various users for infection control; and
at least one zone advertising device for remote zone proximity detection and connection/pairing with the smart face protective devices in accordance with claim 7.

13. The smart zone protection system as claimed in claim 12, wherein the controlled zone is an indoor facility such as a hospital, a school, a conference room, a reception area, an elevator, a building waiting area, a shopping mall, and a corporation hallway.

14. A method of controlling infections within a controlled zone, the method comprising:
configuring at least one zone advertising device as claimed in claim 7 in such a manner to have a strong signal coverage within the entire controlled zone; and
configuring or allowing to be configured a plurality of smart face protective devices associated to various users for enabling remote zone proximity detection and connection/pairing with the at least one zone advertising device.

15. The method of controlling infections within a controlled zone of claim 14, wherein the configuring or allowing to be configured a plurality of smart face protective devices comprise configuring the smart face protective devices with the unique identifiers associated to the at least one zone advertising device and the signal strength threshold.

16. The method of controlling infections within a controlled zone of claim 14, comprising deploying the at least one zone advertising device within the controlled zone configured to emit zone notification signals and configuring the smart face protective devices to automatically secure and lock in place the face shields when said zone notifications signals are detected in accordance with the preconfigured criteria.

17. The method of controlling infections within a controlled zone as claimed in claim 14, comprising:
deploying a first zone advertising device at the entry point of the controlled zone and a second zone advertising device at the exit point of the controlled zone;
configuring the first zone advertising device to emit zone entry point notification signals and configuring the second zone advertising device to emit zone exit point notification signals;
configuring the smart face protective devices to automatically secure and lock in place the face shields when said zone entry point notifications signals are detected in accordance with preconfigured criteria; and
configuring the smart face protective devices to automatically unsecure or unlock the face shields when said zone exit point notifications signals are detected in accordance with preconfigured criteria.
